(19) United States
(12) Patent Application Publication
(10) Pub. No.: US 2005/0115574 A1
(43) Pub. Date: Jun. 2, 2005

(54) Device for supporting the cervical vertebral column

(76) Inventor: Josef Schmitz, Riedering (DE)

Correspondence Address: JACOBSON HOLMAN PLLC, 400 SEVENTH STREET N.W., SUITE 600, WASHINGTON, DC, 20004, US

(21) Appl. No.: 10/513,224

(22) PCT Filed: May 2, 2003
(86) PCT No.: PCT/DE03/01414

(30) Foreign Application Priority Data
May 3, 2002 (DE) 102 19 868.3

(51) Int. Cl. A61B 19/00

(52) U.S. Cl. 128/871; 128/869

(57) ABSTRACT

The invention relates to a device for supporting the cervical vertebral column, in particular during flight in an aircraft. Said device consists of a pressurised cushion comprising at least two front cushions (5, 6), which acts on the region between the head and shoulders of a pilot (1). The helmet (H) of the pilot is provided with a rest piece (4) for the chin. Said piece bridges a gap between the front end regions of the front pressurised cushions (5, 6), covering the upper faces of the front pressurised cushions (5, 6) and can be displaced in relation to the latter, so that the head (1) of the pilot can turn freely and his or her chin is prevented from entering the gap.

The present invention relates to a device for supporting the cervical vertebral column according to the generic term of patent claim 1.

Such a device follows the publication WO/PCT/EP 00/09759. It essentially comprises an assembly of pressurized cushions with at least two front cushions, which face each other in relation to the neck of the user. One the one side these two front cushions rest against the shoulder of a user and on the other side they support the undersides of the lateral lower jaw area of the user. Moreover, the said device has at least one cushion that is positioned in the rear section of the device that rests against the shoulder of the user on one side and supports the occipital squama of the user on the other side. These three cushions exert a pressure in their effective direction for supporting the head in a vertical direction. A control and regulation unit is provided which can selectively raise or lower the pressure in the pressurized cushions. Depending on the tilt of the head and the g-force components measured, this control and regulation unit can build up various countering forces in the effective direction.

A problem of a device of this kind for supporting the vertebral column is that the two front pressurized cushions supporting the undersides of the lateral lower jaw area make it difficult to turn the head even in a zero pressure state when the head is moved toward the front.

In addition, there is the possibility that when the head is tilted toward the front under stronger use of force, the chin of the user may slip through the front areas of both the front pressurized cushions such that the force-compensating function is no longer guaranteed and it is additionally not possible to carry out sideward movements any more.

A further problem of the said invention is the fact that the chin strap of the user's helmet can impair the function of the front cushions considerably. However, a helmet is indispensable.

The task of the present invention is, therefore, to create a device for supporting the vertebral column that allows for freedom of head movement.

This task has been realized by a device having the characteristics of patent claim 1 for supporting the vertebral column.

The main advantage of the present invention is that the provided chin rest pad attached to the chin strap of the helmet provides special support for the head with the chin on the rest pad on one side and support for the rest pad on the front pressurized cushions on the other side, wherein particularly the existing gap between the front end areas of the front pressurized cushions is bridged over by the rest pad. Since the rest pad can easily slide on the front pressurized cushions, it is now possible to move the head conveniently. This also prevents the head from entering into the said gap between the front end areas of the front pressurized cushions and the resulting injury during strong head movements toward the front.

Further advantages of the present invention are that in case of g-loads, the forces affecting the head with the helmet of a pilot are partly compensated on the chin joint and the occipital squama, wherein the front pressurized cushions at the chin joint take over the compensation in case of a head tilt of up to preferably 30 degrees toward the front and up to more than 17 degrees toward the back. The back pressurized cushions at the occipital squama take over the compensation in case of a head tilt from preferably 22 degrees to preferably more than 40 degrees toward the back. The head is not supported in case of an almost upright position from approximately 17 to 22 degrees to the front and to the back and about 5 degrees sideward. The centers of gravity of the head and helmet system almost overlap each other. The relevant g-forces on the vertebral column are moderate in this position of the head since the g-force component is small compared to the countering forces provided by the neck musculature. The specified angular degrees refer to the head position of 0 degrees, in which the center of gravity of the head respective to the mass of helmet/head lies vertically over the fulcrum on the vertebral column.

During operation without a g-load in case of a head tilt a small amount of pressure in the pressurized cushions maintains the cushions upright and against the head. Thus almost full freedom of movement is ensured in case of or with little counter bearing pressure.

In case of a g-load up to 6 g the quantum and the vector of the load in relation to the head position at the moment of this action are recorded by a g-sensor and a control and regulation unit provides a countering force to the cushions for the g load to be counter controlled as a result. Each time, an appropriate control valve actuates the corresponding pressure pillow, till the default bearing pressure at the head is achieved. Force sensors on the surface of the pressurized cushions record the force on the head. This force is compared with a set value in the control and regulation unit and eventually the corresponding control valve is activated to achieve a pressure reduction in the pressurized cushion. In case of an intended movement of the head, the musculature applies an additional small amount of pressure on the force sensors. The control and regulation unit steer the pressure back and thus enable a movement in the intended direction. In case of an acceleration of over 6 g., the control valves to the pressurized cushions are opened for maximum pressure supply and then completely closed. The head is supported in the case of these loads. The target is not to achieve a controlled freedom of movement because this is physically not feasible. The mechanism effectively relieves the cervical vertebral column of stress by up to approximately 60% and the stress on the neck musculature by up to almost 100% depending on the position of the head and the location of the center of gravity.

Normally in cases of a high g-load, injuries of the cervical vertebral column are expected to occur due to a resulting force that amounts to as much as 2.0 times the weight. The device, according to the invention can effectively avoid such injuries. Likewise the invention can prevent stress on the neck musculature that normally leads to exhaustion, blackouts, neurological deficits, coordination disturbances, headaches, partial performance disturbances and a diminished ability to concentrate.

A further advantage of the present invention is that by using the helmet display, displayed images and information can be seen by the pilot vibration free, because the present device stabilizes the head-helmet system.

Dependent claims of this patent further elaborate on the merits of this invention.

The invention and its arrangements are explained in better detail in the context of the following figures. The figures illustrate:

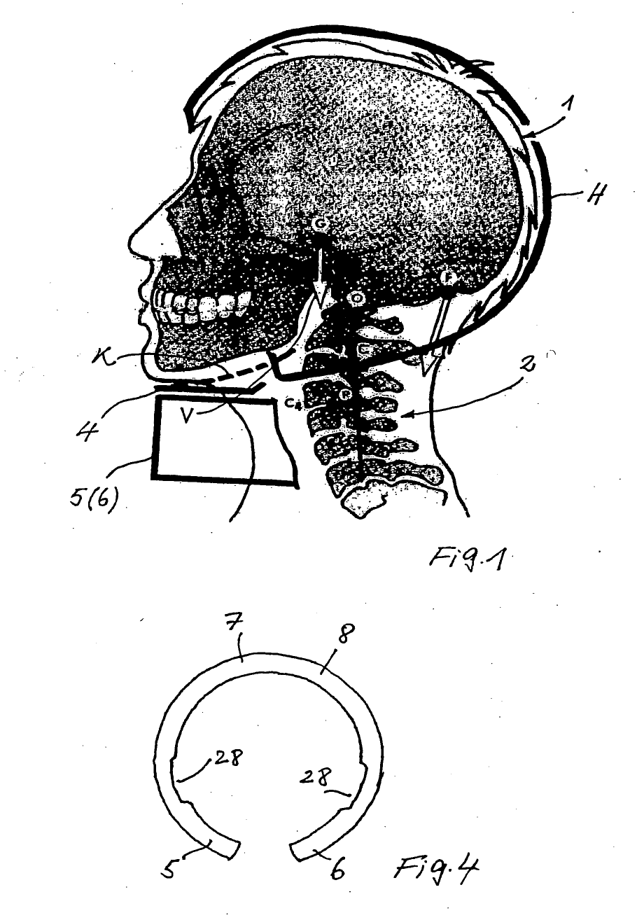

FIG. 1: a schematic representation for the explanation of the invention in which the weight center of the head, the pivot point (fulcrum) between the cervical vertebral column and the head and the application of force on the neck musculature are shown.

FIG. 2: a perspective representation of a preferred pattern of construction of the device according to the invention, essentially consisting of the resting pad attached to the chin strap of a helmet, the front pressurized cushions with hard surface for the adhesion with the head part and the shoulder part with the resting case;

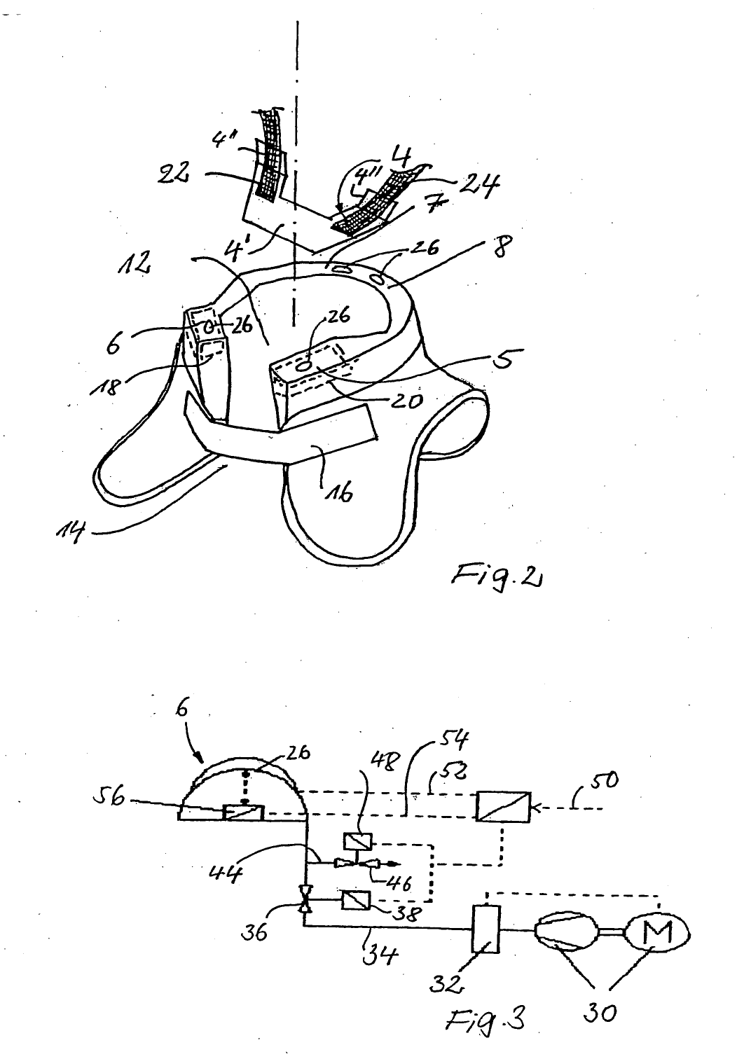

FIG. 3: a schematic block diagram of the control and regulation unit for the explanation of the control of the pressure build-up and pressure reduction in a pressurized cushion; and

FIG. 4: a further training of the invention.

The invention was the result of the following deliberations. According to FIG. 1 the head 1 of a pilot is twisted around the pivot point O with reference to the cervical vertebral column 2. The pivot point O is located at the upper end of the cervical vertebral column 2. The turning takes place through the neck's muscular system that acts on the head 1 in point F. The tilting movement of the head 1 operates in the opposite direction. The tilting movement arises from the weight of the head 1 and tries to turn the head 1 around the pivot point O. It is noticeable that while wearing a helmet, especially one fixed with additional equipment like for instance sight or after-sight devices, the weight of the head 1 and consequently also the tilting moment around the pivot point O are greatly increased. The corresponding vector is indicated in the FIG. 1 with V. In order to avoid the pressure on the head from the front end areas of the front cushions during a large g-load and when the head 1 tilts toward the front, such that the function of the cushion is not affected, according to the invention, a rest pad 4 is attached at the helmet H, favorably at the belt component K of the helmet H on the side turned toward the front cushions 5, 6. This resting pad 4 can glide on the surface of the front cushions 5, 6, in the normal head position indicated in FIG. 1 without a g-load, during a head tilt in case of little pressure in the front cushions 5, 6. Thus an adhesion of the front cushions 5, 6 with the head 1 is maintained. It is also then possible to turn the head conveniently. Almost total freedom of movement can be ensured with minimal countering pressure.

In case of a high g-load (up to 6 g), the quantum and the vector of the force in relation to the head position at the moment of the exposure are recorded by sensors and the control and regulating unit applies a countering force to the cushions that as a result have to be counter controlled till a preset bearing pressure on the head 1 is attained. During an intended movement of the head 1 an additional small amount of pressure is applied by the muscular system to the force sensors of the respective front cushions 5,6, so that the control and regulating unit steers the pressure back and consequently can let a movement in the intended direction. In case of a very big acceleration (over 6 g) the cushions are actuated for a maximum pressure impact, so that the head 1 is fully supported during these stresses. A controlled freedom of movement is not aimed for, because this is not feasible physically. By this mode of operation of the cushions and of the sliding overlying rest pad 4, depending on the position of the head and location of the center of gravity, a reduction of the stress of the cervical vertebral column 2 by up to approximately 60% and a reduction of stress on the neck muscular system by up to almost 100% can be achieved.

According to FIG. 2 the front cushions 5, 6 and the back cushions 7, 8 are fastened at the guard zone of an upper opening 12 of a rest case 10. This rest case 10 overlies on the neck and shoulder area of a pilot. The rest case 10 shows in the forefront a port shaped opening 14 that passes downward which can be widened for putting on the rest case 10, and after that is attachable by drawing together the port shaped opening 14 with the help of at least one locking mechanism 16 running in cross direction which might be a Velcro fastener for instance.

The back cushions 7, 8 lie on the upper side of the occipital squama of the head 1. The front cushions 5, 6 show integrated stiffening elements 18, 20, on which the rest pad 4 can be supported. Each of the cushions 5, 6, 7, 8 is fillable with the help of a fluid, especially a gel, as will be explained in better detail later, so that it can expand in the effective direction to support the head 1 of the pilot. It has been indicated that the cushions 5, 6, 7, 8 can be effectively padded using foam cushion towards the outside, inside and upwards.

If the rest case 10 overlies on the shoulder area of a pilot and the locking 16 is locked, the upper sides of the back cushions 7, 8 lie at the occipital squama of the head 1 of the pilot and the upper sides of the front cushions 5, 6 are turned toward the sideward lower jaw areas of the pilot with their stiffening elements 18, 20. The rest pad 4 is fastened to parts of the chin belt 22, 24 of the helmet H that is not represented in FIG. 2. It is formed such that in the normal state it can overlie on the upper sides of the stiffening elements 18, 20 and/or of the wrapping of the front cushions 5, 6 that covers these, such that it can freely glide on the cushion when the head 1 of the pilot turns. A pushing through of the chin area of the head -1 is avoided, because the rest pad 4 bridges over the gap between the front end areas of the front cushions 5, 6 with its front area 4′. The side areas 4″ are formed stretching obliquely outwards from the front area 4′ and overlie upon the front cushions 5, 6 when the head 1 is upright, so that the sliding motion of the rest pad 4 cannot be obstructed.

A force sensor 26 is placed on the upper side of each cushion 5, 6, 7, 8. It is a piezoresistive pressure sensor that emits an output voltage between 0 Volt and 10 Volt as an output signal. This depends on the pressure applied to it and to the respective cushion.

FIG. 3 shows a schematic block diagram of a control and regulating unit for the production and supply of pressure on each cushion 5, 6, 7, 8, wherein only one cushion 6 has been illustrated in FIG. 3 as an example. A compressor 30 produces the required operating pressure in a reservoir 32. The compressor 30 is connected to the cushion 6 via a duct 34 and a regulating valve 36 for pressure-buildup. The regulating valve 36 is activated via an actuator 38 that is activated by the electronic unit 40. In the duct 34 between the regulating valve 36 and the pressure cushion 6 there is a branch circuit 44 with a discharge valve 46, via which the built up pressure of cushion 6 is relieved with its activation by the actuator 48, which is also activated by the electronic unit 40.

The electronic unit 40 receives an output signal from a 3D-vectorial g-force sensor via the wire 50. This signal contains information about the respective g-force in three-dimensional space. Depending on this signal, the electronic unit 40 produces corrective signals for the pressure supply of the cushion 6 via the regulating valve 36 and/or via the discharge valve 46 for the pressure relief of cushion 6. The pressure sensors 26 of the cushions 5, 6, 7, 8 supply via the wire 52, a pressure signal to the electronic regulating unit 40 indicating the respective pressure in the pressure cushion 6. A range sensor 56 can be provided in order to supply a range signal to the electronic unit 40 via the wire 54. The signal indicates the actually occurring expansion of the cushion 6 after application of pressure via the regulating unit 36.

The front cushions 5, 6 can show on the inner surface in each case indentations 28 in the stiffening elements 18, 20, as illustrated schematically in the FIG. 4. The indentations can be covered by the wrapping and can prevent an injurious squeeze of the carotis communis arteries and of the Ingularis veins. Such indentations do not adversely affect the support of the front cushions 5, 6 on the lower jaw areas in the present invention, because they are covered on the upper sides by the rest pad 4.

The present device can also be used effectively for the compensation of vibration stresses occurring while flying in helicopters and caused by the rotor blades of helicopters where the sinusoidal vibrations of this kind lie in the frequency range of 30 to 100 Hz, and/or in case of sonic frequencies or frequencies of over 300 Hz transmitted by GFK-aircraft cells with an approximately 0.15 oscillating g-acceleration and in case of a longer lasting acceleration of an approximate maximum of 4 g. The vibration stresses originate from either the stimulation of the head by way of the pilot seat and also in case of higher frequencies by the sound pressure of the rotor directly on the helmet via the cabin roof airway. The so-called PIOS (Pilot Induced Oscillations) can also be compensated.

1. Device for supporting the cervical vertebral column, in particular during a flight in a jet aircraft. The device comprises an assembly of pressurized cushions between the head (1) and shoulders of a pilot. The device contains at least two front cushions (5, 6) whose front end areas are distanced from each other by a gap and that there is a provision for a rest pad for the chin of the pilot. The device is characterized by the fact that a rest pad (4) is placed at the helmet (H) of the pilot. The rest pad can be laid out to bridge over the gap existing between the front end areas of the front cushions (5, 6) and can slide with respect to these cushions, so that the head (1) of the pilot can turn freely and his chin does not enter into the gap.

2. Device according to claim 1, characterized by the fact that the front cushions (5,6) are attached at the guard zone of an opening (12) of a rest case (10) lying over the neck and shoulder area of the pilot, such that its upper sides are turned toward the lateral lower jaw areas of the pilot, and that the rest pad (4) can slide smoothly on the upper areas.

3. Device according to claim 1, characterized by the fact that the rest pad (4) is attached to the chin strap (22, 24) of the helmet (H).

4. Device according to claim 1 characterized by the fact that the front cushions (5, 6) show integrated stiffening elements (18, 20) on their upper surfaces, on which the rest pad (4) can be supported.

5. Device according to claim 1, characterized by the fact that the rest pad (4) shows a front area (4′) that bridges over the gap between the front cushions (5, 6). A side area is arranged at each end of the front area (4′) that emerges from the front area (4′) and stretches obliquely outwards.

6. Device according to claim 5 characterized by the fact that the free end areas (4″) of the side areas stretch from the level side area obliquely upwards away from the front cushions (5, 6).

7. Device according to claim 1 characterized by the fact that the front cushions (5, 6) and the back cushions (7, 8) are arranged at the guard zone of the opening (12) of the rest case (10), wherein the surfaces of the back cushions (7, 8) support the occipital squama of the head (1) of the pilot.

8. Device according to claim 7, characterized by the fact that the stiffening elements (18, 20) of the front cushions (5, 6) each show at least one indentation (28) on the inner side, by which a pressure on at least one artery is avoidable.

9. Device according to claim 1 characterized by the fact that the helmet (H) is connected via chin strap components (22, 24) with the rest pad (4).

10. Device according to claim 9 characterized by the fact that the chin belt components (22, 24) are fastened to the side areas of the rest case (4).

11. Device according to claim 2, characterized by the fact that the rest case (10) indicates on the front side a port shaped opening (14) that runs downward away from the front cushions (5, 6) and that can be opened for putting on the rest case (10) and can be locked by a locking mechanism (16).

12. Device according to claim 11, characterized by the fact that the port shaped opening (14) stretches emerging from the gap
